(19) United States
(12) Patent Application Publication
(10) Pub. No.: US 2006/0148796 A1
(43) Pub. Date: Jul. 6, 2006

(54) 6-Substituted imidazopyrazines

(76) Inventors: Peter Jan Zimmermann, Radolfzell (DE); Wilm Buhr, Konstanz (DE); M. Vittoria Chiesa, Konstanz (DE); Andreas Palmer, Konstanz (DE); Christof Brehm, Konstanz (DE); Gerhard Grundler, Konstanz (DE); Joerg Senn-Bilfinger, Konstanz (DE); Wolfgang-Alexander Simon, Konstanz (DE); Stefan Postius, Konstanz (DE); Wolfgang Kromer, Konstanz (DE)

Correspondence Address: NATH & ASSOCIATES PLLC, 112 South West Street, Alexandria, VA, 22314, US

(21) Appl. No.: 10/545,190

(22) PCT Filed: Feb. 16, 2004
(86) PCT No.: PCT/EP04/50135

§ 371 Date: Nov. 9, 2005

(30) Foreign Application Priority Data
Feb. 18, 2003 (EP) 03003652.9

(51) Int. Cl. A61K 31/5377; A61K 31/498; C07D 487/04

(52) U.S. Cl. 514/234.5; 514/249; 544/331; 544/117

(57) ABSTRACT

The present invention relates to 6-substituted imidazopyrazines of formula 1 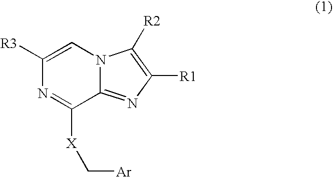 in which the substituents and symbols have the meanings indicated in the description. The compounds have gastric secretion inhibiting and excellent gastric and intestinal protective action properties.

### TECHNICAL FIELD

The invention relates to novel compounds, which are used in the pharmaceutical industry as active compounds for the production of medicaments.

### PRIOR ART

In European patent applications 204285 (which corresponds to U.S. Pat. Nos. 4,725,601 and 4,782,055), certain imidazoheterocyclic compounds are disclosed, which are said to be useful in the treatment of ulcers. In European patent application 299470 (which corresponds to U.S. Pat. No. 5,112,834), certain imidazopyridines and —pyrazines are disclosed, which are said to have an excellent protective effect on the stomach and intestine of warm-blooded animals. International patent application WO 99/28322 (which corresponds to U.S. Pat. No. 6,518,270) describes heterocyclic compounds, among others imidazopyrazines with a certain substitution pattern, which are said to inhibit gastric acid secretion. In J. Med. Chem. 1987, 30, 2031-2046, J. Kaminski et al. describe the gastric antisecretory, cytoprotective and metabolic properties of certain substituted imidazo[1,2-a]pyrazines, which are unsubstituted in the 6-position. In ‘The Practice of Medicinal Chemistry’, pages 203-237, C. Wermuth gives a review on ‘Molecular Variations based on isosteric Replacements’. In the abstract of Japanese Patent publication No. 07242666, a variety of heterocyclic compounds is disclosed, amongst which imidazo[l,2-a]pyridines are exemplarily named, which are said to be useful for preventing and treating allergy, inflammation, autoimmune diseases, shock, ache, etc., as bradykinine-antagonizing agents. In International patent application WO 021060492 certain (condensed) pyridine and pyrazine derivatives are disclosed, which are said to be useful as kinase inhibitors.

### SUMMARY OF THE INVENTION

The invention relates to compounds of the formula 1 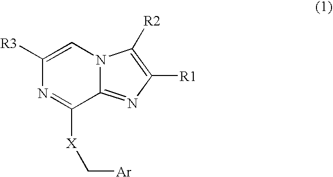
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, hydroxy-1-4C-alkyl, fluoro-1-4C-alkyl, halogen, 2-4C-alkenyl, 2l-4C-alkynyl, amino, mono- or di-1-4C-alkylamino-1-4C-alkyl or cyanomethyl,
R3 is halogen, fluoro-1-4C-alkyl, 2-4C-alkenyl, 2-4-C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R31R32,
   where
   R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4alkyl,
   or where
   R31 and R32 together, including the nitrogen atom to which both are bonded, are a pyrrolidino, piperidino, piperazino, N-1-4C-alkylpiperazino or morpholino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R4 is hydrogen, 1-4-alkyl, hydroxy-1-4C-alkyl, 1-4-C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-1C-alkoxycarbonylamino or sulfonyl,
      in which
      aryl is phenyl or substituted phenyl with one, two or three identical or different substituents from the group of 1-4-C-alkyl, 1-4C-alkoxy, carboxy, 1-4C-alkoxycarbonyl, halogen, trifluoromethyl, nitro, trifluoromethoxy, hydroxy and cyano,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl, 1-4C-alkoxy, hydroxy or halogen and
   R7 is hydrogen, 1-4C-alkyl or halogen,
      and the salts of these compounds.

1-4C-Alkyl represents straight-chain or branched alkyl groups having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents a group, which in addition to the oxygen atom contains one of the aforementioned 1-4C-alkyl groups. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (1-4C-alkoxy-C(O)—) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents straight-chain or branched alkenyl groups having 2 to 4 carbon atoms. Examples which may be mentioned are the 2-butenyl, 3-butenyl, 1-propenyl and the 2-propenyl group (alkyl group).

2-4C-Alkynyl represents straight-chain or branched alkynyl groups having 2 to 4 carbon atoms. Examples which may be mentioned are the 2-butynyl, 3butynyl, and preferably the 2-propynyl, group (propargyl group).

Fluoro-1-4C-alkyl represents one of the aforementioned 1-4C-alkyl groups, which is substituted by one or more fluorine atoms. An example which may be mentioned is the trifluoromethyl group.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3hydroxypropyl group.

Halogen within the meaning of the invention is bromo, chloro and fluoro.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

Mono- or di-1-4C-alkylamino-1-4C-alkyl represents a 1-4C-alkyl group, which is substituted by a mono- or di-1-4C-alkylamino group. Examples which may be mentioned are the dimethylaminomethyl, the diethylaminomethyl, the methytaminomethyl and the dilsopropylaminomethyl group.

1-4C-Alkoxy-1-4C-alkoxy represents one of the aforementioned 1-4C-alkoxy groups, which is substituted by a further 1-4C-alkoxy group. Examples which may be mentioned are the groups 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. An example which may be mentioned is the group 2-(methoxy)ethoxymethyl (CH₃—O—CH₂—CH₂—O—CH₂—).

Fluoro-1-4C-alkoxy represents one of the aforementioned 1-4C-alkoxy groups, which is completely or mainly substituted by fluorine, “mainly” meaning in this connection that more than half of the hydrogen atoms are replaced by fluorine atoms. Examples of completely or mainly fluoro-substituted 1-4C-alkoxy groups which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoro-ethoxy, in particular the 1,1,2,2-tetrafluoroethoxy, the 1,2,2-trifluoro-ethoxy, the trifluoromethoxy and preferably the difluoromethoxy group

Fluoro-1-4C-alkoxy-1-4C-alkyl represents one of the aforementioned 1-4C-alkyl groups, which is substituted by a fluoro-1-4C-alkoxy group. Examples of fluoro-1-4C-alkoxy-1-4C-alkyl groups are the 1,1,2,2-tetrafluoroethoxymethyl, the 2,2,2-trifluoroethoxymethyl, the trifluoromethoxyethyl and the difluoromethoxyethyl group.

1-7C-Alkyl represents straight-chain or branched alkyl groups having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl and the methyl group.

Groups Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl-3-methyl-2-pyrrolyl, 5-ethoxycarbonyl-2,4-dimethyl-3-pyrrolyl, 3,4-dibromo-5methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoro methylphenyl)-3-pyrrolyl, 1-(2,6dichloro-4-trifluoromethylphenyl)-2-pyrrolyl, 1-(2-nitrobenzyl)-2-pyrrolyl, 1-(2-fluorophenyl-2-pyrrolyl, 1-(4-trifluoromethoxyphenyl)-2-pyrrolyl, 1-(2-nitrobenzyl)-2-pyrrolyl, 1-(4ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxy-carbonyl-1-(2,6-dichlorophenyl)-5-pyrazolyl, 5-allyloxy-1-methyl-3trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3indolyl, 1-benzyl-3-indolyl, 2-(4-chlorophenyl)-3-indolyl, 7-benzyloxy-3-indolyl, 6-benzyloxy-3-indolyl, 2-methyl-nitro-3-indolyl, 4,5,6,7-tetrafluoro-3-indolyl, 1-3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4nitro-5-isoxazolyl, 2-pyridyl, 3-pyridyl, 4-pyridyl, 6-methyl-2-pyridyl, 3-hydroxy-5-hydroxymethyl-2-methyl-4pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6methyl-4-phenyl-3-pyridyl, 2-chloro-3-pyridyl, 6-(3-trfluoromethylphenoxy)-3-pyridyl, 2-(4-chlorophenoxy)-3-pyridyl, 2,4-dimethoxy-5-pyrimidinyl, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

2-4C-Alkenyloxy represents groups, which in addition to the oxygen atom contain one of the abovementioned 2-4C-alkenyl groups. Examples, which may be mentioned, are the 2-butenyloxy, 3-butenyloxy, 1-propenyloxy and the 2-propenyloxy group (alkyloxy group).

1-4C-Akylcarbonyl represents a group, which in addition to the carbonyl group contains one of the aforementioned 1-4C-alkyl groups. An example which may be mentioned is the acetyl group.

Carboxy-1-4C-alkyl represents 1-4C-alkyl groups which are substituted by a carboxyl group. Examples, which may be mentioned, are the carboxymethyl and the 2-carboxyethyl group.

1-4C-Alkoxycarbonyl-1-4C-alkyl represents 1-4C-alkyl groups, which are substituted by one of the abovementioned 1-4C-alkoxycarbonyl groups. Examples, which may be mentioned, are the Methoxycarbonylmethyl and the ethoxycarbonylmethyl group.

Aryl-1-4C-alkyl represents one of the aforementioned 1-4C-alkyl groups, which is substituted by one of the abovementioned aryl groups. An exemplary preferred aryl -1-4C-alkyl group is the benzyl group.

Aryl-1-4C-alkoxy represents one of the aforementioned 1-4C-alkoxy groups, which is substituted by one of the abovementioned aryl groups. An exemplary preferred aryl-1-4C-alkoxy group is the benzyloxy group.

1-4C-Alkylcarbonylamino represents an amino group to which a 1-4C -alkycarbonyl group is bonded. Examples which may be mentioned are the propionylamino (C₃H₇C(O)NH—) and the acetylamino group (acetamido group) (CH₃C(O)NH—).

1-4l-C Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-(methoxy)ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-4C-alkoxy-1-4C-alkoxycarbonyl groups. Examples which may be mentioned are the 2-(methoxy)ethoxycarbonylamino and the 2-(ethoxy)ethoxycarbonylamino group.

Possible salts of compounds of the formula 1—depending on substitution—are especially all add addition salts. Particular mention may be made of the pharmacologically tolerable salts of the inorganic and organic adds customarily used in pharmacy. Those suitable are water-soluble and water-insoluble acid addition salts with acids such as, for example, hydrochloric acid, hydrobromic add, phosphoric acid, nitric acid, sulfuric add, acetic acid, citric acid, D-gluconic acid, benzoic add, 2-(4-hydroxybenzoyl)benzoic add, butyric acid, sulfosalicylic acid, maleic acid, lauric add, malic add, fumaric add, succinic add, oxalic acid, tartaric add, embonic add, stearic acid, toluenesulfonic acid, methanesulfonic acid or 3-hydroxy-2-naphthoic acid, where the acids are used in salt preparation—depending on whether a mono- or polybasic add is concerned and on which salt is desired—in an equimolar quantitative ratio or one differing there from.

Pharmacologically intolerable salts, which can initially be obtained, for example, as process products in the production of the compounds according to the invention on the industrial scale, are converted into the pharmacologically tolerable salts by processes known to the person skilled in the art.

It is known to the person skilled in the art that the compounds according to invention and their salts, if, for example, they are isolated in crystalline form, can contain various amounts of solvents. The invention therefore also comprises all solvates and in particular all hydrates of the compounds of the formula 1, and also all sonates and in particular all hydrates of the salts of the compounds of the formula 1.

One embodiment of the invention (embodiment a) relates to compounds of the formula 1a 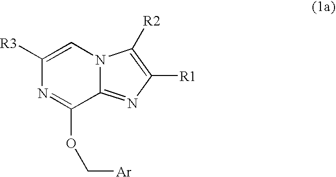
in which R1, R2, R3 and Ar have the meanings given above, and their salts.

Another embodiment of the invention (embodiment b) relates to compounds of the formula 1b 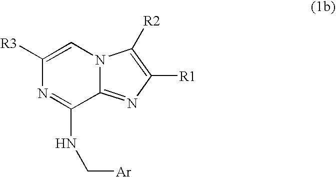
in which R1, R2, R3 and Ar have the meanings given above, and their salts.

Among the compounds of formula 1, those are to be mentioned particularly, in which
R1 is hydrogen or 1-4C-alkyl,
R2 is hydrogen or 1-4C-alkyl,
R3 is halogen, carboxyl, 1-4C-alkoxycarbonyl, hydroxy-1-4C-alkyl, 1-4C-alkoxy-1-4C-alkyl, or the group —CO—NR31R32,
   where
   R31 is hydrogen, 1-7C-alkyl, hydroxy-1-4C-alkyl or 1-4C-alkoxy-1-4C-alkyl and
   R32 is hydrogen or 1-7C-alkyl,
   or where
   R31 and R32 together, including the nitrogen atom to which both are bonded, are a pyrrolidino, piperidino, piperazino, N-1l-4C-alkylpiperazino or morpholino group,
X is O (oxygen) or NH,
Ar is a phenyl group substituted in the 2-position by R4 and in the 6-position by R5,
   where
   R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
   R5 is hydrogen, 1-4C-alkyl or 1-4C-alkoxy,
   or
   Ar is selected from the group consisting of 4-acetoxyphenyl, 4-acetamidophenyl, 2-methoxyphenyl, 3-methoxyphenyl, 4-methoxyphenyl, 3-benzyoxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-fluorophenyl, 2-chloro-5-nitrophenyl, 4-chloronitrophenyl, 3-(4-chloro-3phenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-3-indolyl, 1-methyl-2-(4-trifluorophenoxy)-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4pyridyl, 3-chloro-5-trifluoromethyl-2-pyridyl, 4,6-dimethyl-2-pyridyl, 4-(4chlorophenyl)-3pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3l-pyridyl, 2,4-dimethoxy-5-pyrimidinyl, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl,
      and the salts of these compounds.

Among the compounds of the formula 1, those of the formula 1-1 have to be highlighted 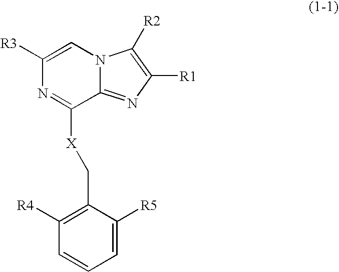
in which
R1 is hydrogen or 1-4C-alkyl,
R2 is hydrogen or 1-4C-alkyl,
R3 is halogen, carboxyl, 1-4C-Alkoxycarbonyl, hydroxy-1-4C-alkyl, 1-4C-alkoxy-1-4C-alkyl or the group —CO—NR31R32
   where
   R31 is hydrogen, 1-7C-alkyl, hydroxy-1-4C-alkyl or 1-4C-alkoxy1-4C-alkyl and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino or morpholino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
   and the salts of these compounds.

Compounds of embodiment a to be highlighted are those of formula 1-1, in which
R1 is hydrogen or 1-4C-alkyl,
R2 is hydrogen or 1-4C-alkyl,
R3 is halogen, carboxyl, 1-4C-alkoxycarbonyl, hydroxy-1-4C-alkyl, 1-4C-alkoxy-1-4-C-alkyl or the group —CO—NR31R32
   where
   R31 is hydrogen, 1-7C-alkyl, hydroxy-1-4C-alkyl or 1-4C-alkoxy-1-4C-alkyl and
   R32 is hydrogen or 1-7C-alkyl,
   or where
   R31 and R32 together, including the nitrogen atom to which both are bonded, are a pyrrolidino, piperidino, piperazino, N1-4C-alkylpiperazino or morpholino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen),
   and the salts of these compounds.

Compounds of embodiment b to be highlighted are those of formula 1-1, in which
R1 is hydrogen or 1-4C-alkyl,
R2 is hydrogen or 1-4C-alkyl,
R3 is halogen, carboxyl, 1-4C-alkoxycarbonyl, hydroxy-1-4C-alkyl, 1-4C-alkoxy-1-4C-alkyl or the group —CO—NR31R32
   where
   R31 is hydrogen, 1-7C-alkyl, hydroxy-1-4C-alkyl or 1-4C-alkoxy-1-4C-alkyl and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino or morpholino group,
R4 is hydrogen, 1-4-C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is NH,
   and the salts of these compounds.

Compounds of the formula 1-1 to be emphasised are those, in which
R1 is 1-4C-alkyl,
R2 is 1-4C-alkyl,
R3 is carboxyl, 1-4C-alkoxycarbonyl, hydroxy-1-4C-alkyl, 1-4C-alkoxy-1-4C-alkyl or the group —CO—NR31R32,
   where
   R31 is hydrogen, 1-7C-alkyl, hydroxy-1-4C-alkyl or 1-4C-alkoxy-1-4C-alkyl and
      R32 is hydrogen or 1-7C-alkyl,
   or where
   R31 and R32 together, including the nitrogen atom to which both are bonded, are a pyrrolidino, piperidino, piperazino, N-1-4C-alkylpiperazino or morpholino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
   and the salts of these compounds.

Preferred compounds of the formula 1-1 are those, in which
R1 is 1-4C-alkyl,
R2 is 1-4C-alkyl,
R3 is carboxyl, 1-4C-alkoxycarbonyl), hydroxy-1-4C-alkyl, 1-4C-alkoxy-1-4C-alkyl or the group —CO— NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl,
R4 is 1-4C-alkyl or 1-4C-alkylcarbonylamino,
R5 is 1-4C-alkyl and
X is O (oxygen) or NH,
   and their salts.

Particularly preferred compounds of the formula 1-1 are those, in which
R1 is 1-4C-alkyl,
R2 is 1-4C-alkyl,
R3 is carboxyl 1-4C-alkoxycarbonyl, hydroxy-1-4C-alkyl, 1-4C-alkoxy-1-4C-alkyl or the group —CO— NR31R32,
   where
   R31 is hydrogen, 1-4C-alkyl or hydroxy-1-4C-alkyl and
   R32 is hydrogen or 1-4C-alkyl,
R4 is 1-4C-alkyl,
R5 is 1-4C-alkyl and
X is O (oxygen) or NH,
   and their salts.

Particularly preferred are the compounds given as final products of formula 1 in the examples, and the salts of these compounds.

The compounds according to the invention can be synthesised from corresponding starting compounds, for example according to the reaction schemes given below. The synthesis is carried out in a manner known to the expert, for example as described in more detail in the following examples.

According to the invention, the compounds of formula I can be prepared as outlined in the reaction schemes 1 and 2, which illustrate processes known to the expert and which use known starting materials. The particular method for the synthesis and reaction sequence of the compounds of formula 1 is chosen having regard to the specific nature of the substituents and their position. One of the processes for producing the compounds of formula 1 consists in condensing a 3,5-disubstituted 2-aminopyrazine II with an alpha-halocarbonylcompound III (scheme 1). The required 3,5-disubstituted 2-aminopyrazines II are obtained by a substitution reaction of a 5-substituted 2-amino-3-bromopyrazine I, containing any desired substituent R3, with ArCH₂XH(X═O or NH), analogously to known procedures (see, for example, EP 204285).

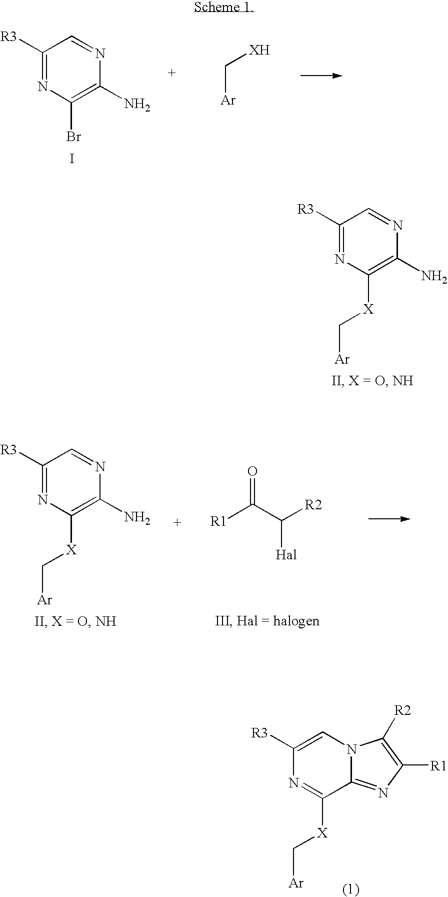

Another process (scheme 2) for producing the compounds of formula 1 consists in carrying out a substitution reaction starling with an appropriate substituted imidazo[1,2-a]pyrazine IV or V). It is thus possible, for example, starting from compounds of formula IV, to prepare compounds of formula 1 where R2=CH₂OH (e.g. by Vilsmeler reaction and subsequent reduction) or where R2=Br or Cl (by bromination or chlorination). Further derivatizaton of compounds of formula 1, where R2=Br or Cl, can be accomplished for example by metal-catalysed carbonylation to get compounds of formula 1 where R2=alkoxycarbonyl or by using the Sonogashira reaction to get compounds of formula 1 where R2=propynyl. A substitution of R3 can be carried out likewise, for example by palladium-catalysed carbonylation of compounds V as described in more detail in the examples. If compounds, where R3=—CO—NR31R32, are desired, an appropriate derivatization can be performed in a manner known per se (conversion of an ester or carboxylic acid into an amide).

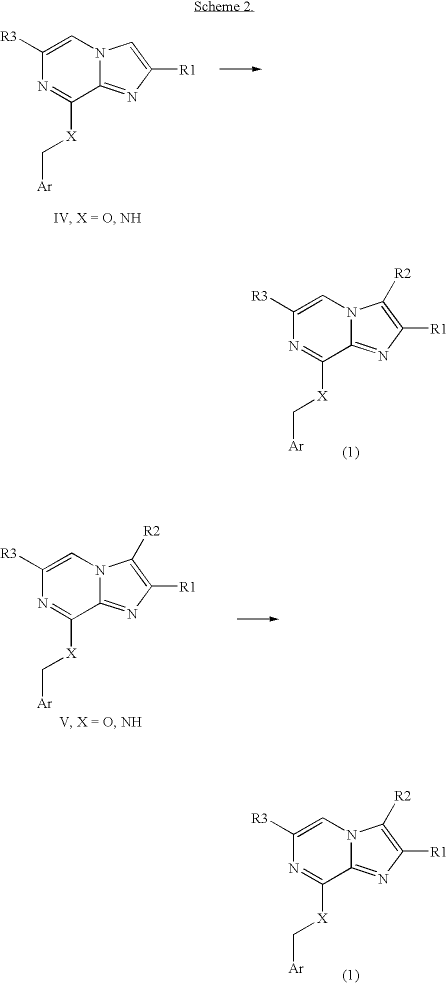

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation min stands for minute(s), h for hour(s). The compounds under the scope of formula 1 named expressly as examples, and any salts of these compounds, are preferred subject matter of the invention.

### EXAMPLES

### 1. 2-Ethyl-6-methyl-benzylamine

To a suspension of 10.4 g (275 mmol) of lithium aluminium hydride in 200 ml of dried diethyl ether is slowly added a solution of 20.0 g (138 mmol) of 2-ethyl-6-methyl-benzonitrile in 60 ml of diethyl ether at −10° C. After 1 h at 0° C. and 1 h at room temperature, the reaction mixture is carefully hydrolyzed with 4 ml of water and 4 ml of 6N sodium hydroxide solution. After 2 h at mom temperature, anhydrous magnesium sulfate is added and the reaction mixture is filtered through Celite. Evaporation of the solvent yields 15.5 g (80%) of the title compound as a colourless oil which is used without further purification in the next step.

### 2. 2-Amino-5-bromo-3-(2-ethyl-6-methyl-benzylamino)-pyrazine

A solution of 1.26 g (5 mmol) of 2-amino-3,5-dibromopyrazine (B. Jiang at al., Bioorg. Med. Chem. 2001, 9, 1149-1154), 1.5 g (10 mmol) of 2-ethyl-6-methyl-benzylamine and 1.5 ml of triethylamine in 3.5 ml of acetonitrile in a sealed tube is irradiated in a microwave-oven for 40 min (temperature 180° C.). The crude reaction mixtures of 10 such runs are combined, treated with saturated sodium hydrogen carbonate and extracted with ethyl acetate. The organic phase is dried over anhydrous magnesium sulfate and evaporated. The residue is purified by column chromatography on silica gel using light petroleum ether:ethyl acetate (4:1, v/v). Crystallization from dioxane yields 10.2 g (68%) of the title compound as a colorless solid (m.p. 155° C.).

### 3. 6-Bromo-8(2-ethyl-6-methyl-benzylamino)-2,3-dimethyl-imidazo[1,2-a]pyrazine oxalate

To a suspension of 10.0 g (31 mmol) of 2-amino-5-bromo-3-(2-ethyl-6-methyl-benzylamino)-pyrazine in 60 ml of dioxane are added 4.9 ml (46.7 mmol) of 3-bromo-2-butanone and the resulting mixture is heated to 100° C. After 2 h a further amount of 4.9 ml (46.7 mmol) of 3-bromo-2-butanone is added and the mixture is stirred for 16 h. The mixture is cooled down, diluted with dichloromethane and extracted with saturated aqueous sodium hydrogen carbonate. The organic phase is dried over anhydrous magnesium sulfate and evaporated. Purification of the residue by column chromatography on silica gel using light petroleum ether/ethyl acetate (4:1, v/v) gives a colourless oil which is dissolved in acetone and treated with a solution of 3.91 g (31 mmol) of oxalic acid dihydrate in acetone. The precipitate is collected and washed with n-heptane to yield 10 g (70%) of the title compound as a colourless solid (m.p. 163° C.).

### 4. Ethyl 8-(2-ethyl-6-methyl-benzylamino)-2,3-dimethyl-imidazo[1,2-a]pyrazine-6-carboxylate

10.0 g (22 mmol) of 6-bromo-8-(2-ethylmethyl-benzylamino-2,3-dimethylmidazo[1,2-a]pyrazine oxalate are treated with saturated aqueous sodium hydrogen carbonate and extracted with ethyl acetate. The organic phase is separated, dried over anhydrous magnesium sulfate and evaporated to give 6-bromo 2-ethyl-6-methyl-benzylamino)-2,3-dimethyl-imidazo[1,2-a]pyrazine as a colourless oil. The resulting oil thus obtained is dissolved in 80 ml of absolute ethanol and 16 ml of triethylamine and transferred to an autoclave. After addition of 0.5 g (2.2 mmol) of palladium(II) acetate and 1.64 g (6.2 mmol) of triphenylphosphine, the reaction mixture is carbonylated (10 bar carbon monoxide pressure, 100° C.) for 14 h. The reaction mixture is cooled down, filtered and evaporated to leave an orange coloured oil which is dissolved in dichloromethane and extracted with water. The organic phase is dried over anhydrous magnesium sulfate and evaporated. Purification of the residue by crystallization from ethyl acetate/n-heptane yields 7.2 g (89%) of the title compound as a colourless solid (m.p. 144° C.).

### 5. 6-(Dimethylaminocarbonyl)-8-(2-ethyl-6-methyl-benzylamino)-2,3-dimethyl-imidazo[1,2a]-pyrazine

To a solution of 2.3 g (5.1 mmol) of 6-bromo-8-(2-ethyl-6-methyl-benzylamino)-2,3-dimethyl-imidazo[1,2-a]pyrazine in 50 ml of dimethylamine (2M solution in tetrahydrofuran) are added 0.17 g (0.76 mmol) of palladium(II) acetate and 0.8 g (3.1 mmol) of triphenylphosphine. The mixture is transferred to an autoclave and carbonylated (6 bar carbon monoxide pressure, 120° C.) for 16 h. The reaction mixture is cooled down, evaporated and the residue dissolved in dichloromethane. The organic phase is extracted with saturated aqueous ammonium chloride solution, dried over anhydrous magnesium sulfate and evaporated. Purification of the residue by column chromatography on silica gel using light petroleum ether/ethyl acetate (1:1, v/v) yields 1.22 g (66%) of the title compound as a colourless solid (m.p. 174° C.).

### 6. 8-(2-Ethyl-6-methyl-benzylamino)-2,3-dimethyl-imidazo[1,2-a ]pyrazine-6-carboxylic acid

To a solution of 4.0 g (10.9 mmol) of ethyl 8-(2-ethyl-6-methyl-benzylamino)- 2,3-dimethyl-imidazo[1,2-a]pyrazine-6-carboxylate in 40 ml of dioxane are added 8 ml of 2N aqueous sodium hydroxide solution. After 1 h at 80° C., the reaction mixture is evaporated to half of its volume and the pH is adjusted to 6 by the addition of 6N hydrochloric acid. The thick precipitate is collected, washed with water and dried in vacuo over phosphorus pentoxide to yield 3.52 g (95%) of the title compound as a colourless solid (m.p. 230° C.).

### 7. 8-(2-Ethyl-6-methyl-benzylamino)-6-(pyrrolidinocarbonyl)-2,3-dimethyl-imidazo[1,2-a]pyrazine

To a suspension of 0.5 g (1.48 mmol) of 8-(2-ethyl-6-methyl-benzylamino-2,3-dimethyl-imidazo(1,2-a]pyrazine-6-carboxylic add in 10 ml of dichloromethane are added 0.7 g (2.2 mmol) of O-(1H-benzotriazol-1-yl)-N,N,N′,N′-tetramethyluronium tetrafluoroborate (TBTU). After 30 min 0.5 ml (6 mmol) of pyrrolidine are added and the mixture is stirred for 7 h. The reaction mixture is extracted with 2N aqueous sodium hydroxide solution, the organic phase is separated, dried over anhydrous magnesium sulfate and evaporated. Purification of the residue by column chromatography on silica gel using dichloromethane/methanol (20:1, v/v) and crystallization from ethyl acetate/n-heptane yields 0.45 g (78%) of the the compound as a colourless solid (m.p. 197° C.).

### 8. 8-(2-Ethyl-6-methyl-benzylamino)-2,3dimethyl-imidazo[1,2-a]pyrazine-6-carboxamide

To a suspension of 1.02 g (3 mmol) of 8-(2-ethyl-6-methyl-benzylamino)-2,3-dimethyl-imidazo[1,2-a]pyrazine-6-carboxylic acid in 20 ml of dichloromethane are added 1.61 g (5 mmol) of O-(1H -benzotriazol-1-yl)-N,N,N′,N′-tetramethyluronium tetrafluoroborate (TBTU). After 30 min ammonia gas is passed over the mixture. After 1 h, a further amount of 1.0 g (3.1 mmol) of TBTU is added. Stirring is continued for 1 h at room temperature and finally 1 h under reflux. The reaction mixture is extracted with 2N aqueous sodium hydroxide solution, the organic phase is separated, dried over anhydrous magnesium sulfate and evaporated. Purification of the residue by column chromatography on silica gel using dichloromethane/methanol (20:1, v/v) and crystallization from ethyl acetate/n-heptane yields 0.67 g (66%) of the title compound as a colourless solid (map. 227° C.).

### 9. 8-(2-Ethyl-6-methyl-benzylamino)-6-(methylaminocarbonyl)-2,3-dimethyl-imidazo[1,2a]-pyrazine

To a suspension of 1.02 g (3 mmol) of 8-(2-ethyl-6-methyl-benzylamino)-2,3-dimethyl-imidazo[1,2-a]-pyrazine-6-carboxylic acid in 20 ml of dichloromethane are added 1.61 g (5 mmol) of O-(1H-benzotriazol-1-yl)-N,N,N′,N′-tetramethyluronium tetrafluoroborate (TBTU). After 30 min stirring at room temperature 1 ml (8 mmol) of methylamine (8M in ethanol) is added. After 1 h, a further amount of 0.5 ml (4 mmol) of methylamine (8M in ethanol) is added and stirring is continued for 16 h. The reaction mixture is extracted with 2N aqueous sodium hydroxide solution, the organic phase is separated, dried over anhydrous magnesium sulfate and evaporated. Purification of the residue by column chromatography on silica gel using ethyl acetate/light petroleum ether (1:1, v/v) and crystallization from ethyl acetate/n-heptane yields 0.99 g (94%) of the tile compound as a colourless solid (m.p. 120° C.).

### 10. 8-(2-Ethyl-6-methyl-benzylamino)-6-(2-methoxyethylaminocarbonyl-2,3-dimethyl-imidazo[1,2-a]pyrazine

A solution of 1.0 g (2.73 mmol) of ethyl 8-(2-ethyl-6methyl-benzylamino)-2,3-dimethyl-imidazo[1,2-a]pyrazine-6-carboxylate in 10 ml of 2-methoxyethylamine is heated under reflux for 20 h. The reaction mixture is diluted with water and extracted with dichloromethane. The organic phase is dried over anhydrous magnesium sulfate and evaporated. Purification of the residue by column chromatography on silica gel using ethyl acetate/light petroleum ether (1:1, v/v) and crystallization from diisopropyl ether yields 0.53 g (49%) of the title compound as a colourless solid (m.p. 111° C.).

### 11. 8-(2-Ethyl-6-methyl-benzylamino)-6-(2-hydroxyethylaminocarbonyl)-2,3-dimethylimidazo[1,2a]pyrazine

A suspension of 1.1 g (3 mmol) of ethyl 8-(2-ethyl-8-methyl-benzylamino)-2,3-dimethyl-imidazo[1,2-a]pyrazine-6-carboxylate in 10 ml of 2-aminoethanol is heated to 80° C. for 30 min. The reaction mixture is diluted with an additional amount of 10 ml of 2-aminoethanol and the temperature is raised to 100° C. After 1 h, the reaction mixture is cooled down and the precipitate is collected and washed with water. The colourless solid is dried in vacuo over phosphorus pentoxide to yield 1.04 g (91%) of the title compound (m.p. 229° C.).

### 12. 8-(2-Ethyl-6-methyl-benzylamino)-6-(hydroxymethyl)-2,3-dimethyl-imidazo[1,2-a]pyrazine

To a suspension of 0.31 g (82 mmol) of lithium aluminium hydride in 10 ml of dried tetrahydrofuran is slowly added a solution of 1.0 g (2.7 mmol) of ethyl 8-(2-ethyl-6-methyl-benzylamino)-2,3-dimethyl-imidazo[1,2-a]pyrazine-6-carboxylate in 20 ml of tetrahydrofuran at 0° C. After 1 h at 0° C., the reaction mixture is carefully hydrolyzed with 0.2 ml of water, 0.4 ml of 6N sodium hydroxide solution and 1 ml of water. After 1 h at room temperature, anhydrous magnesium sulfate is added and the reaction mixture is filtered through Celite. On evaporation of the filtrate, a precipitate is obtained which is washed with diethyl ether and dried in vacuo to yield 0.77 g (87%) of the title compound as a colourless solid (m.p. 166° C.).

### 13. 8-(2-Ethyl-6-methyl-benzylamino)-6-(methoxymethyl)-2,3-dimethyl-imidazo[1,2-a]pyrazine hydrochloride

To a suspension of 0.5 g (1.54 mmol) of 8-(2-ethyl-6-methyl-benzylamino)-6-hydroxymethyl-2,3-dimethyl-imidazo[1,2-a]pyrazine in 5 ml of absolute N,N-dimethylformamide are added 0.18 g (4.5 mmol) of sodium hydride (60% w/w dispersion in mineral oil) in portions at room temperature. After 30 min, 0.12 ml (1.95 mmol) of methyliodide are slowly added. After 30 min, the reaction mixture is carefully hydrolyzed with saturated aqueous sodium hydrogen carbonate solution and extracted with dichloromethane. The organic phase is dried over anhydrous magnesium sulfate and evaporated. Purification of the residue by column chromatography on silica gel using ethyl acetate/light petroleum ether (1:4, v/v) yields 0.18 g of a colourless oil which is dissolved in dichloromethane and treated with hydrogen chloride (1.5M in diethyl ether). Evaporation of all volatiles yields 0.12 g (21%) of the title compound as a colourless solid (m p. 177° C.).

Commercial Utility

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or add regurgitation.

In their excellent properties, the compounds according to the invention surprisingly prove to be dearly superior to the compounds known from the prior art in various models in which the antiulcerogenic and the antisecretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the abovementioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several preferably 1 to 4, individual doses to achieve the desired result in the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquilizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino adds.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, meziocillin, ampicillin, amoxicillin, cefalothin, cefoxitin, cefotaxime, imipenem, gentamycin, amikacin, erythromycin, ciprofloxacin, metronidazole, clariothromycin, azithromycin and combinations thereof (for example clarithromycin+metronidazole).

In view of their excellent gastric and intestinal protection action, the compounds of formula 1 are suited for a free or fixed combination with those medicaments (e.g. certain antiinflammatories and antirheumatics, such as NSAIDs), which are known to have a certain ulcerogenic potency. In addition, the compounds of formula 1 are suited for a free or fixed combination with motility-modifying drugs.

Pharmacology

The excellent gastric protective action and the gastric add secretion-inhibiting action of the compounds according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds according to the invention on the pentagastrin-stimulated add secretion of the perfused rat stomach after intraduodenal administration in vivo is shown.

TABLE A

| | Dose | Inhibition of |
|---|---|---|
| Example | (μmol/kg) | acid secretion |
| No. | i.d. | (%) |
| 5 | 1 | 49 |
| 13 | 1 | 46 |

Methodology

The abdomen of anesthetized rats (CD rat, female, 200-250 g; 1.5 g/kg Im. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (.e. after determination of 2 preliminary fractions). The substances to be tested were administered intraduodenally in a 2.5 ml/kg liquid volume

60 min after the start of the continuous pentagastrin infusion.

The body temperature of the animals was kept at a constant 37.8-38° C. by infrared irradiation and heat pads (automatic, stepless control by means of a rectal temperature sensor).

1. A compound of the formula 1 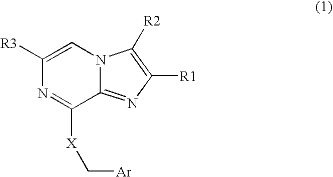
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, hydroxy-1-4C-alkyl, fluoro-1-4C-alkyl, halogen, 2-4C-alkenyl, 2-4C-alkynyl, amino, mono- or di-1-4C-alkylamino-1-4C-alkyl or cyanomethyl,
R3 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
where
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino or morpholino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
in which
aryl is phenyl or substituted phenyl with one, two or three identical or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl, 1-4C-alkoxy, hydroxy or halogen and
R7 is hydrogen, 1-4C-alkyl or halogen,
or a solvate, hydrate, salt, hydrate of a salt or solvate of a salt thereof.

2. A compound of formula 1 according to claim 1, in which X is O (oxygen) or a solvate, hydrate, salt, hydrate of a salt or solvate of a salt thereof.

3. A compound of formula 1 according to claim 1, in which X is NH or a solvate, hydrate, salt, hydrate of a salt or solvate of a salt thereof.

4. A compound of the formula 1 according to claim 1, characterized by the formula 1-1 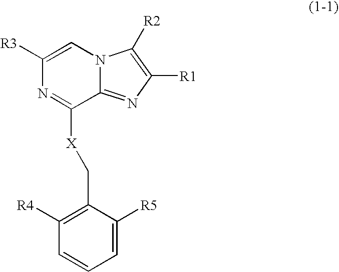
in which
R1 is hydrogen or 1-4C-alkyl,
R2 is hydrogen or 1-4C-alkyl,
R3 is halogen, carboxyl, 1-4C-alkoxycarbonyl, hydroxy-1-4C-alkyl, 1-4C-alkoxy-1-4C-alkyl or the group —CO—NR31R32,
where
R31 is hydrogen, 1-7C-alkyl,.hydroxy-1-4C-alkyl or 1-4C-alkoxy-1-4C-alkyl and
R32 is hydrogen or 1-7C-alkyl,
or where
R31 and R32 together, including the nitrogen atom to which both are bonded, are a pyrrolidino, piperidino, piperazino, N-1-4C-alkylpiperazino or morpholino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
or a solvate, hydrate, salt, hydrate of a salt or solvate of a salt thereof.

5. A compound of the formula 1-1 according to claim 4, in which
R1 is 1-4C-alkyl,
R2 is 1-4C-alkyl, R3 is carboxyl, 1-4C-alkoxycarbonyl, hydroxy-1-4C-alkyl, 1-4C-alkoxy-1-4C-alkyl or the group —CO—NR31R32,
where
R31 is hydrogen, 1-7C-alkyl, hydroxy-1-4C-alkyl or 1-4C-alkoxy-1-4C-alkyl and
R32 is hydrogen or 1-7C-alkyl,
or where
R31 and R32 together, including the nitrogen atom to which both are bonded, are a pyrrolidino, piperidino, piperazino, N-1-4C-alkylpiperazino or morpholino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
or a solvate, hydrate, salt, hydrate of a salt or solvate of a salt thereof.

6. A compound of the formula 1-1 according to claim 4, in which
R1 is 1-4C-alkyl,
R2 is 1-4C-alkyl,
R3 is carboxyl, 1-4C-alkoxycarbonyl, hydroxy-1-4C-alkyl, 1-4C-alkoxy-1-4C-alkyl or the group —CO—NR31R32,
where
R31 is hydrogen, 1-4C-alkyl, hydroxy-1-4C-alkyl or 1-4C-alkoxy-1-4C-alkyl and
R32 is hydrogen or 1-4C-alkyl,
‘R4 is 1-4C-alkyl or 1-4C-alkylcarbonylamino,
R5 is 1-4C-alkyl and
X is O (oxygen) or NH,
or a solvate, hydrate, salt, hydrate of a salt or solvate of a salt thereof.

7. A compound of the formula 1-1 according to claim 4, in which
R1 is 1-4C-alkyl,
R2 is 1-4C-alkyl,
R3 is carboxyl, 1-4C-alkoxycarbonyl, hydroxy-1-4C-alkyl, 1-4C-alkoxy-1-4C-alkyl or the group —CO—NR31R32,
where
R31 is hydrogen, 1-4C-alkyl or hydroxy-1-4C-alkyl and
R32 is hydrogen or 1-4C-alkyl,
R4 is 1-4C-alkyl,
R5 is 1-4C-alkyl and
X is O (oxygen) or NH,
or a solvate, hydrate, salt, hydrate of a salt or solvate of a salt thereof.

8. A compound according to claim 4, in which X is NH, or a solvate, hydrate, salt, hydrate of a salt or solvate of a salt thereof.

9. A pharmaceutical composition comprising a compound as claimed in claim 1 and/or a pharmacologically acceptable solvate, hydrate, salt, hydrate of a salt or solvate of a salt thereof together with a suitable pharmaceutical auxiliary and/or excipient.

10. (canceled)

11. (canceled)

12. A method of treating or preventing a gastrointestinal disorder in a patient comprising administering to a patient in need thereof a therapeutically effective amount of a compound of claim 1 or a pharmacologically acceptable solvate, hydrate, salt, hydrate of a salt or solvate of a salt thereof.

13. A compound according to claim 5, in which X is NH, or a solvate, hydrate, salt, hydrate of a salt or solvate of a salt thereof.

14. A compound according to claim 6, in which X is NH, or a solvate, hydrate, salt, hydrate of a salt or solvate of a salt thereof.

15. A compound according to claim 7, in which X is NH, or a solvate, hydrate, salt, hydrate of a salt or solvate of a salt thereof.
